SEQUENCE LISTING (39 sequences)

SEQ ID NO: 1 - DNA, 627 bp, Homo sapiens
  CDS (55)..(504)
gcggccgcaagctcggcactcacggctctgagggctccgacggcactgacggccatggcgcgttcgaacctcccgctggcgctgggcctggccctggtcgcattctgcctcctggcgctgccacgcgacgcccgggcccggccgcaggagcgcatggtcggagaactccgggacctgtcgcccgacgacccgcaggtgcagaaggcggcgcaggcggccgtggccagctacaacatgggcagcaacagcatctactacttccgagacacgcacatcatcaaggcgcagagccagctggtggccggcatcaagtacttcctgacgatggagatggggagcacagactgccgcaagaccagggtcactggagaccacgtcgacctcaccacttgccccctggcagcaggggcgcagcaggagaagctgcgctgtgactttgaggtccttgtggttccctggcagaactcctctcagctcctaaagcacaactgtgtgcagatgtgataagtccccgagggcgaaggccattgggtttggggccatggtggagggcacttcaggtccgtgggccgtatctgtcacaataaatggccagtgctgcttcttgcaaaaaaaaaaaaaaaaaaa

SEQ ID NO: 2 - protein, 149 aa, Homo sapiens
MARSNLPLALGLALVAFCLLALPRDARARPQERMVGELRDLSPDDPQVQKAAQAAVASYNMGSNSIYYFRDTHIIKAQSQLVAGIKYFLTMEMGSTDCRKTRVTGDHVDLTTCPLAAGAQQEKLRCDFEVLVVPWQNSSQLLKHNCVQM

SEQ ID NO: 3 - DNA, 251 bp, Homo sapiens
tgctggggactgtcgctgtcctctccctccccgggccaggtgtgtcctggagggcagggaagcgtcttggcacgcgggtgcgcgccgccccctcggcctcctgggctccctgaacctcgcaggaccccggcaacttcgagccccgccccagctccaggccgcgggggcgcatcgcgggcgtcgggcggggcggcccagcgggtaaaagctgcgcggccgcaagctcggcactcacggctctgagggctccg

SEQ ID NO: 4 - DNA, 24 bp, Homo sapiens
tgctggggactgtcgctgtcctct

SEQ ID NO: 5 - DNA, 24 bp, Homo sapiens
gcactcacggctctgagggctccg

SEQ ID NO: 6 - DNA, 15 bp, Homo sapiens
gctgagcctggccct

SEQ ID NO: 7 - DNA, 17 bp, Homo sapiens
ccgctgggcctgaccct

SEQ ID NO: 8 - DNA, 21 bp, Homo sapiens
cgctggcgctgagcctggccc

SEQ ID NO: 9 - DNA, 22 bp, Homo sapiens
cgctgtgactatgaggtccttg

SEQ ID NO: 10 - DNA, 15 bp, Homo sapiens
actatgaggtctttg

SEQ ID NO: 11 - DNA, 21 bp, Homo sapiens
cgggcgtcggcggggcggccc

SEQ ID NO: 12 - DNA, 22 bp, Homo sapiens
gggccgggtgtcccctcccagc

SEQ ID NO: 13 - DNA, 21 bp, Homo sapiens
gacccctgacctgcccctacc

SEQ ID NO: 14 - DNA, 21 bp, Homo sapiens
ggagggctggggctggaggag

SEQ ID NO: 15 - DNA, 22 bp, Homo sapiens
ggtcgaggctgggctcacccct

SEQ ID NO: 16 - DNA, 22 bp, Homo sapiens
ggggcagaagcgaagcagttgg

SEQ ID NO: 17 - DNA, 19 bp, Homo sapiens
ggagaactccgggacctgt

SEQ ID NO: 18 - DNA, 20 bp, Homo sapiens
cagtctgtgctccccatctc

SEQ ID NO: 19 - DNA, 22 bp, Homo sapiens
gtacttcctgacgatggagatg

SEQ ID NO: 20 - DNA, 21 bp, Homo sapiens
taggagctgagaggagttctg

SEQ ID NO: 21 - DNA, 24 bp, Homo sapiens
gtcgccctggacttcgagcaagag

SEQ ID NO: 22 - DNA, 22 bp, Homo sapiens
ctagaagcatttgcggtggacg

SEQ ID NO: 23 - DNA, 36 bp, Homo sapiens
cggccgcaggagcgcacggtcggagaactccgggac

SEQ ID NO: 24 - DNA, 36 bp, Homo sapiens
gtcccggagttctccgaccgtgcgctcctgcggccg

SEQ ID NO: 25 - DNA, 36 bp, Homo sapiens
tgtgactttgaggtctttgtggttccctggcagaac

SEQ ID NO: 26 - DNA, 36 bp, Homo sapiens
gttctgccagggaaccacaaagacctcaaagtcaca

SEQ ID NO: 27 - DNA, 35 bp, Homo sapiens
ggtccttgtggttcccgcgcagaactcctctcagc

SEQ ID NO: 28 - DNA, 35 bp, Homo sapiens
gctgagaggagttctgcgcgggaaccacaaggacc

SEQ ID NO: 29 - DNA, 32 bp, Homo sapiens
ctgagctctgctggggactgtcgctgtcctct

SEQ ID NO: 30 - DNA, 32 bp, Homo sapiens
atctcgagcggagccctcagagccgtgagtgc

SEQ ID NO: 31 - DNA, 20 bp, Homo sapiens
taagagcccacgaagagctg

SEQ ID NO: 32 - DNA, 20 bp, Homo sapiens
ccttgatgatgtgcgtgtct

SEQ ID NO: 33 - DNA, 20 bp, Homo sapiens
catttatttcctgccggtgt

SEQ ID NO: 34 - DNA, 20 bp, Homo sapiens
ccacttgagccaaggagttc

SEQ ID NO: 35 - DNA, 20 bp, Homo sapiens
ctcccaaagtgctgggatta

SEQ ID NO: 36 - DNA, 20 bp, Homo sapiens
gagggctgtccagaatgaac

SEQ ID NO: 37 - DNA, 25 bp, Homo sapiens
ggttttttgggttttttgaattttg

SEQ ID NO: 38 - DNA, 24 bp, Homo sapiens
taccaaacttacaaccacacaact

SEQ ID NO: 39 - DNA, 24 bp, Homo sapiens
taccgaacttacgaccgcgcaact

(19) United States
(12) Patent Application Publication
(10) Pub. No.: US 2011/0098242 A1
(43) Pub. Date: Apr. 28, 2011

(54) Methods and Compositions for Diagnosing and Treating Cervical Intraepithelial Neoplasia and Cervical Cancer

(76) Inventors: Eri S. Srivatsan, Montrose, CA (US); Mysore S. Veena, Los Angeles, CA (US)

(21) Appl. No.: 12/993,068

(22) PCT Filed: May 18, 2009
(86) PCT No.: PCT/US2009/044287

§ 371 Date: Nov. 16, 2010

Related U.S. Application Data
(60) Provisional application No. 61/054,349, filed on May 19, 2008.

(51) Int. Cl. A61K 31/706; C12Q 1/68; G01N 33/574; C07H 21/04; A61K 31/165; A61P 35/00

(52) U.S. Cl. 514/43; 435/6; 435/7.23; 536/23.1; 514/575

(57) ABSTRACT

Disclosed herein are methods and compositions for diagnosing and treating diseases associated with a loss of cystatin E/M expression including cervical intraepithelial neoplasia and cervical cancer. Genetic mutations and exonic deletions which result in a loss of cystatin E/M expression are also disclosed.

### CROSS-REFERENCE TO RELATED APPLICATIONS

This application claims the benefit of U.S. Patent Application Ser. No. 61/054,349, filed 19 May 2008, which is herein incorporated by reference in its entirety.

### ACKNOWLEDGEMENT OF GOVERNMENT SUPPORT

This invention was made with Government support of Grant No. W0333, awarded by the VA Merit Review. The Government has certain rights in this invention.

### BACKGROUND OF THE INVENTION

1. Field of the Invention

The present invention generally relates to diagnosing and treating cervical intraepithelial neoplasia to cervical cancer in a subject.

2. DESCRIPTION OF THE RELATED ART.

Cervical cancer is the second most common cancer responsible for cancer related death in women around the world. The incidence is increasing, with 450,000 new cases diagnosed annually worldwide. The disease is frequently found in women having multiple sex partners, smoking habits, and immune system dysfunctions. Cervical cancer is closely linked with human papilloma virus (HPV) infection and HPVs are detected in greater than 90% of cervical cancer lesions. Close to 100 different HPV types are described in cervical cancers with HPV 16 and HPV 18 being commonly associated with these tumors. HPVs play an important role in tumorigenesis by inactivating TP53 and retinoblastoma (RB) tumor suppressor genes. However, detailed studies on a large number of tumors indicate that although HPV is present, viral infection may not be sufficient for tumor development. Studies have shown that 20-30 million Americans are infected with HPV, but only a subset of them develop cervical cancer.

Thus, a need exists for methods and compositions for diagnosing and treating cervical intraepithelial neoplasia and cervical cancer.

### SUMMARY OF THE INVENTION

The present invention provides an isolated nucleic acid molecule which (a) specifically binds to a nucleotide region having at least 15 and up to 40 (specifically including 17, 20, 21, 22, 24, 25, 32, 35, and 36) nucleotide bases, and (a1) a nucleotide sequence or its complement having SEQ ID NO:1 with at least one nucleotide mutation selected from the group consisting of A185G, G91A, T437A, A374G, T155C, and C445T, or (a2) a genomic sequence which corresponds to the nucleotide sequence or its complement; (b) encodes at least 5 and up to 12, preferably 5 to 8, contiguous amino acid residues of an amino acid sequence having SEQ ID NO:2 with at least one amino acid change selected from the group consisting of D44G, A13T, F128Y, H107R, M34T, and L131F; (c) specifically binds to a primer sequence that (c1) comprises at least 15 and up to 40 (specifically including 17, 20, 21, 22, 24, 25, 32, 35, and 36) contiguous nucleotide bases of SEQ ID NO:1, a genomic sequence which corresponds to SEQ ID NO:1, or a complement thereof, and (c2) is adjacent to the nucleotide region, or a sequence indicative of encoding SEQ ID NO:1 with at least one amino acid deletion. In some embodiments, the sequence of the nucleic acid molecule is not SEQ ID NO:1 or SEQ ID NO:3. In some embodiments, the sequence of the nucleic acid molecule is selected from the group consisting of SEQ ID NO:4 to SEQ ID NO:39. The nucleic acid molecules in accordance with the present invention may be used as primers or probes.

In some embodiments, the present invention provides a panel which comprises at least one nucleic acid molecule as disclosed herein.

In some embodiments, the present invention provides a kit comprising at least one nucleic acid molecule or the panel as disclosed herein.

In some embodiments, the present invention is directed to an expression profile which provides the expression levels of cystatin E/M in normal tissue, pre-neoplastic tissue, and cancerous tissue. In some embodiments, the expression profile may further include the expression levels of cathepsin L in normal tissue, pre-neoplastic tissue, and cancerous tissue. In some embodiments, the tissue is cervical tissue. In some embodiments, the tissue is ovarian tissue.

In some embodiments, the present invention provides a method of diagnosing a subject as having a cancer or identifying a tissue of being cancerous which comprises assaying the loss of cystatin E/M expression in a biological sample obtained from the subject or the tissue which comprises (a) detecting the presence of a nucleotide mutation in a cystatin E/M gene obtained from the biological sample which corresponds to nucleotide position 91, 155, 185, 374, 437, or 445 of SEQ ID NO:1, or an exonic deletion; (b) detecting the presence of an amino acid change in a cystatin E/M protein obtained from the biological sample which corresponds to amino acid position 13, 34, 44, 107, 128, or 131 of SEQ ID NO:2, or a deletion of one or more amino acid residues; (c) comparing the amount of the cystatin E/M protein or the amount of mRNA which encodes the cystatin E/M protein obtained from the biological sample with a control or a standard; (d) detecting hypermethylation of the promoter of the cystatin E/M gene obtained from the biological sample; (e) detecting the presence of a Human Papilloma Viral protein in the biological sample; or a combination thereof, wherein a loss cystatin E/M expression of about 80-100%, preferably about 90-100%, more preferably about 95-100% as compared to a control or standard is indicative of the subject having the cancer or the tissue being cancerous.

In some embodiments, the cancer is cervical cancer and the biological sample or tissue is cervical tissue or the cancer is ovarian cancer and the biological sample or tissue is ovarian tissue. In some embodiments, the subject is suspected of having the cancer. In some embodiments, the subject is human.

In some embodiments, the subject is suspected of having the cancer. In some embodiments, a subject may be classified of being at risk for suffering from the cancer.

The methods of the present invention may further comprise detecting the expression of cathepsin L in the biological sample or tissue sample. In some embodiments, at least about expression of cathepsin L at levels (e.g. more than about 100%, preferably about 150%, more preferably about 200%) above normal expression levels is indicative of the tissue being cancerous tissue. In some embodiments, little to no expression (e.g. expression levels consistent with expression levels in normal tissue) of cathepsin L and a cystatin E/M expression level that is less than (e.g. about 90-10%, preferably about 80-10%, more preferably about 70-10%, most preferably about 60-10%) normal or standard expression levels is indicative of the tissue being pre-neoplastic tissue.

In some embodiments, the present invention provides a method of assaying the loss, if any, of cystatin E/M expression in a biological sample which comprises (a) detecting the presence of a nucleotide mutation in a cystatin E/M gene obtained from the biological sample which corresponds to nucleotide position 91, 155, 185, 374, 437, or 445 of SEQ ID NO:1; (b) detecting the presence of an amino acid change in a cystatin E/M protein obtained from the biological sample which corresponds to amino acid position 13, 34, 44, 107, 128, or 131 of SEQ ID NO:2; (c) comparing the amount of the cystatin E/M protein and/or the amount of mRNA which encodes the cystatin E/M protein obtained from the biological sample with a control or a standard; (d) detecting hypermethylation of the promoter of the cystatin E/M gene obtained from the biological sample; (e) detecting the presence of a Human Papilloma Viral protein in the biological sample; or a combination thereof

In some embodiments, the present invention provides a method of treating a subject suspected of having a cancer which comprises (a) diagnosing the subject as having the cancer according to any one of claims 1 to 4; and (b) administering a treatment to the subject accordingly. Treatments include those which are known in the art. In some embodiments, the effect of the treatment is monitored by assaying the amount of cancerous tissue in the subject and/or measuring the amount of cystatin E/M protein in a sample obtained from the subject.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1A is a schematic map of the human cystatin E/M gene and its exons (Veena et al. (2008) Genes Chromosomes and Cancer 47:740-754, which is herein incorporated by reference). See Homo sapiens chromosome 11 genomic contig, reference assembly showing 1.89 kb region from base 11085067 to 11086960, Accession No. NT_{—}033903.7, GI:51468814, which is herein incorporated by reference. A genomic sequence which corresponds to a cystatin E/M gene includes Accession No. NT_{—}033903.7, GI:51468814.

FIG. 1B shows the sequence of the human cystatin mRNA (SEQ ID NO:1) and the encoded amino acid sequence (SEQ ID NO:2). The underlined portion indicates the homozygous deletion of exon 1. The nucleotides and amino acid residues in bold face represent nucleotide mutations (above) and amino acid changes (below) as set forth in Table 1 (FIG. 4A).

FIG. 2 shows the results of a growth inhibition assay of cells transfected with cystatin E/M. Cells transfected with lipofectamine (L) alone and pCMV vector (CMV) were used as controls.

FIG. 3A shows expression of cystatin E/M in normal tissues and normal cervix.

FIG. 3B shows the loss of cystatin E/M expression in various cell lines and 11 primary cervical tumors. All of the uninvolved normal tissues have high level expression of cystatin E/M. LCK-SCC, SmCC, SCC and AC represent large cell and small cell keratinizing squamous cell carcinoma, squamous cell carcinoma and adenocarcinoma, respectively.

FIG. 4A shows Table 1 which summarizes the cystatin E/M mutations in cervical tumors.

FIG. 4B schematically represents cystatin E/M derived from a protein sequence alignment to cystatin F reported by Schuttelkopf et al. (2006) J Biol Chem 28:16570-16575, which is herein incorporated by reference. As shown, tumors #59 and #62 have mutations in the cathepsin L binding domains, tumor #53 is mutated one amino acid proximal to the consensus site, and tumor #54 has a homozygous deletion of sequences from exon 1. Numbers underneath the arrows represent the location of amino acids affected by point mutations in the tumors and the amino acid conversions are listed in brackets. The dashed line indicates homozygous deletion of exon 1 in tumor #54.

FIG. 4C is a graph showing the results of the promoter assay showing an increased relative luciferase activity in the HeLa cells containing the promoter DNA.

FIGS. 5A and 5B show reduced expression of cystatin E/M keratinocytes transformed with the HPV E7 gene.

FIG. 5A is a Western blot revealing reduced expression of cystatin E/M in keratinocytes transformed with the HPV E7 gene in comparison to that of normal (HEK) and keratinocytes transformed with the HPV E6 gene.

FIG. 5B is a picture of immunofluorescence staining which confirms reduced expression in keratinocytes transformed with the HPV E7 gene at 100× magnification. The inset for a single cell shows uniform expression in keratinocytes transformed with the HPV E6 gene and punctated and reduced staining (signal intensity) in keratinocytes transformed with the HPV E7 gene.

FIG. 6 shows the binding of HPV E7 protein to cystatin E/M. Hybridization of the immnuprecipitates to the cystatin E/M antibody shows presence of cystatin E/M in cells cotransfected with HPV 16 E7, thereby confirming the interaction between the E7 and cystatin E/M proteins. Western blotting of an aliquot of the protein lysate prior to immunprecipitation with β-tubulin indicated the use of equal quantity of protein in the immunoprecipitation reactions.

FIG. 7 shows photomicrographs of normal skin and cervix. The photomicrographs depict the localization and staining characteristics of cystatin E/M and cathepsin L in normal skin and cervix. The top two photomicrographs are from a normal skin with epidermis (panel A) and sebaceous gland (panel B). The second row photomicrographs are from a normal cervix with epithelium (panel C) and endocervical gland (panel D). Panels A-D show cells stained for cystatin E/M. The staining is in the nuclei with frequent perinuclear localizations (arrows). In addition, less prominent cystoplasmic reactions are also observed. Panels E (epithelium) and F (endocervix) are from a normal cervix stained for cathepsin L. Normal epithelial cells have no staining but the endocervical gland cells show fine granular cytoplasmic reactions concentrated near the secreting lumens (arrow) and none with the nuclear presentation. All photographs were obtained with a 60× oil objective lens.

FIG. 8 shows the expression of cystatin E/M in the preneoplastic cervical intraneoplasia lesions (CINs). The photomicrographs are of a cervix showing the epithelium (EP) and endocervical gland (EN) with CIN III involvement (arrow). Cystatin E/M stains are predominantly in the nuclei of the cells (panel A) while cathepsin L expression is observed in the cytoplasm of the endocervical gland cells (panel B). Both photomicrographs are taken with a 40× objective lens.

FIG. 9 shows composite pictures which represent hybridization to two different antibodies, cystatin E/M and cathepsin L, and two different magnifications of a tumor of cervix (#55) with cystatin E/M and cathepsin L immunohistochemical stains. The photomicrographs on the left show cystatin E/M expression and the photomicrographs on the right side show cathepsin L expression. The top row of photomicrographs were taken with a 10× objective lens and the bottom row of photomicrographs are of the same tumor shown in the top row taken with a 40× objective lens. Tumor #55 has minimal or no expression of cystatin E/M but high expression of cathepsin L as depicted in the bottom row (40× objective) of the photomicrographs. As shown in the top left panel, cystatin E/M protein is highly expressed in the superficial epithelium with CIN III pathology (inset) but not in the invasive tumor (T). As shown in the FIG. 9, cells with cathepsin L expression extend into the interstitial tissues of tumor #55.

FIG. 10 is a graph showing the statistically significant difference of cystatin E/M expression in CINs and invasive tumors.

FIG. 11 is a graph showing the statistical significance for the joint score of reactivity X intensity of cystatin E/M expression in CINs versus the tumors. p value=0.0007, Fisher's exact=0.003.

FIG. 12 is a graph showing the near statistical significance (p value=0.04, Fisher exact test score=0.16) observed for the joint reactivity X intensity of cathepsin L expression in the stroma of CINs versus the aggressive tumors.

### DETAILED DESCRIPTION OF THE INVENTION

The present invention relates to compositions and methods for cancer diagnostics and therapies. In particular, the present invention relates to the cystatin E/M (also known as “CST6”) gene and its expression product for diagnosing and treating a subject suspected of having, suffering from, or at risk for suffering from a cancer associated with abnormal cystatin E/M expression, such as cervical cancer and ovarian cancer.

As used herein, the term “subject” refers to any mammal including humans and animals. In some embodiments, the subject is a human subject.

As used herein, a subject “suspected of having” a cancer refers to a subject that presents one or more symptoms indicative of a cancer (e.g., a noticeable lump or mass), a subject to be screened for a cancer (e.g., during a routine physical), a subject having one or more risk factors for a cancer, a subject who has received an initial diagnosis (e.g., a CT scan showing a mass) but has yet to receive test results which confirm the presence of a cancer, and a subject who is in remission.

As used herein, a subject “at risk for” suffering from a cancer refers to a subject having one or more risk factors, such as gender, age, genetic predisposition, environmental expose, previous incidents of cancer, preexisting non-cancer diseases, lifestyle, and the like as known in the art, for developing a given cancer.

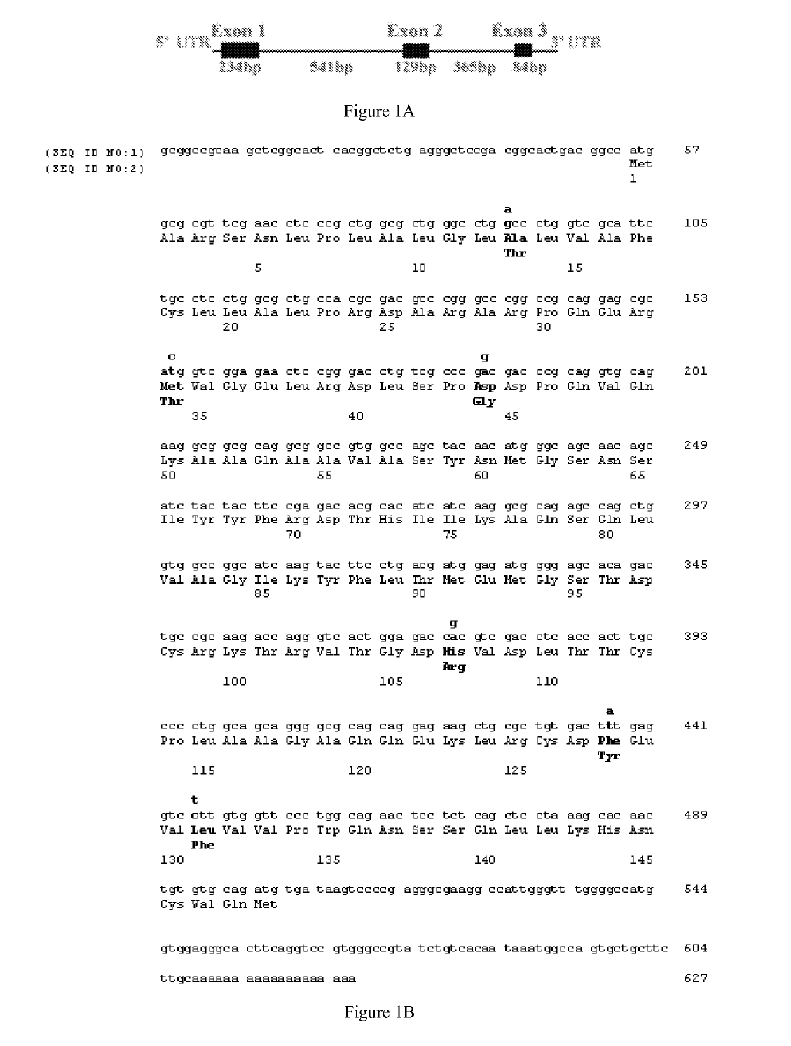

FIG. 1A is a schematic map of the human cystatin E/M gene and its exons. See Sotiropoulou et al. (1997) J. Biol. Chem. 272(2):903-910, which is herein incorporated by reference. The cystatin E/M gene encodes a cystatin E/M (or CST6) protein that is a lysosomal cysteine protease inhibitor (mRNA=SEQ ID NO:1); polypeptide=SEQ ID NO:2). See FIG. 1B. As provided herein, mutations in the cystatin E/M gene which are associated with a loss of cystatin E/M expression have been identified. Probes which specifically bind to polynucleotide sequences which encode these mutations may be used to detect and diagnose abnormal cystatin E/M expression and cancers related abnormal cystatin E/M expression, such as cervical cancer and ovarian cancer. The primers and probes of the present invention are useful in the diagnosis and characterization of CINs and cervical cancer and tumors and other cancerous and precancerous tissues associated with a loss of cystatin E/M expression.

As used herein, “nucleic acid molecule”, “polynucleotide”, and “oligonucleotide” are used interchangeably to refer DNA and RNA molecules of natural or synthetic origin which may be single-stranded or double-stranded, and represent the sense or antisense strand. The nucleic acid molecules of the present invention may contain known nucleotide analogs or modified backbone residues or linkages, and any substrate that can be incorporated into a polymer by DNA or RNA polymerase. Examples of such analogs include phosphoborothioates, phosphoramidates, methyl phosphonates, chiral-methyl phosphonates, 2-O-methyl ribonucleotides, peptide-nucleic acids (PNAs), and the like.

In preferred embodiments, the nucleic acid molecule of the present invention is isolated. As used herein, “isolated” refers to a nucleic acid molecule that is isolated from its native environment. An “isolated” nucleic acid molecule may be substantially isolated or purified from the genomic DNA of the species from which the nucleic acid molecule was obtained. An “isolated” polynucleotide may include a nucleic acid molecule that is separated from other DNA segments with which the nucleic acid molecule is normally or natively associated with at either the 5′ end, 3′ end, or both.

The nucleic acid molecules of the present invention may be in its native form or synthetically modified. The nucleic acid molecules of the present invention may be single-stranded (coding or antisense) or double-stranded, and may be DNA (genomic, cDNA or synthetic) or RNA molecules. RNA molecules include mRNA molecules, which contain introns and correspond to a DNA molecule in a one-to-one manner, and mRNA molecules, which do not contain introns. The nucleic acid molecules of the present invention may be linked to other nucleic acid molecules, support materials, reporter molecules, quencher molecules, or a combination thereof. Other nucleic acid molecules include promoters, polyadenylation signals, additional restriction enzyme sites, multiple cloning sites, other coding segments, and the like. It is therefore contemplated that a nucleic acid fragment of almost any length may be employed, with the total length preferably being limited by the ease of preparation and use in the intended recombinant DNA or PCR protocol.

The nucleic acid molecules of the present invention may be readily prepared by methods known in the art, for example, directly synthesizing the nucleic acid sequence using methods and equipment known in the art such as automated oligonucleotide synthesizers, PCR technology, recombinant DNA techniques, and the like.

The nucleic acid molecules of the present invention may contain a label. A wide variety of labels and conjugation techniques are known by those skilled in the art and may be used in various nucleic acid and amino acid assays employing the nucleic acid molecules of the present invention. As used herein a “label” or a “detectable moiety” is a composition that is detectable by spectroscopic, photochemical, biochemical, immunochemical, or chemical means. A “labeled” nucleic acid molecule comprises a bound label such that the presence of the nucleic acid molecule may be detected by detecting the presence of the label bound to thereto. The label may be bound to the nucleic acid molecule via a covalent bond, such as a chemical bond, or a noncovalent bond, such as ionic, van der Waals, electrostatic, or hydrogen bonds. Methods known in the art for producing labeled hybridization or PCR probes for detecting sequences related to polynucleotides may be used and include oligolabeling, nick translation, end-labeling or PCR amplification using a labeled nucleotide, and the like, preferably end-labeling. Suitable reporter molecules and quencher molecules that may be used include radionucleotides, enzymes, fluorescent, chemiluminescent, or chromogenic agents as well as substrates, cofactors, inhibitors, magnetic particles, and the like. In preferred embodiments, a fluorescent reporter molecule and quencher molecule are used.

As used herein, a “probe” is a molecule that specifically binds to a target molecule, e.g. a nucleic acid probe which specifically binds to a target sequence. As used herein, a “nucleic acid probe” or “oligonucleotide probe” refers to a nucleic acid molecule that is capable of binding to a given nucleic acid molecule (target sequence) having a sequence that is complementary to the sequence of the nucleic acid probe. A nucleic acid probe may include natural or modified bases known in the art. See e.g. MPEP 2422, 8^{th} ed., which is herein incorporated by reference. The nucleotide bases of the nucleic acid probe may be joined by a linkage other than a phosphodiester bond, so long as the linkage does not interfere with the ability of the nucleic acid molecule to bind a complementary nucleic acid molecule. The nucleic acid probe may bind a target sequence that is less than 100% complementary to the nucleic acid probe sequence and such binding depends upon the stringency of the hybridization conditions. The presence or absence of a probe may be detected to determine the presence or absence of a target molecule in a sample. A probe may contain a label whose signal is detectable by methods known in the art. As used herein a “signal” is a characteristic that is measurable using methods known in the art. Where the label is a reporter molecule and a quencher molecule, the signal may increase or decrease upon dissociation of reporter molecule and the quencher molecule. For example, if the reporter molecule is a fluorophore, separation of the quencher from the fluorophore will generate a detectable signal due to an increase in light energy emitted by the fluorophore in response to illumination.

Primers and probes according to the present invention can be designed using, for example, a computer program such as OLIGO (Molecular Biology Insights, Inc., Cascade, CO). Important features when designing oligonucleotides to be used as amplification primers include an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). As with oligonucleotide primers, oligonucleotide probes usually have similar melting temperatures, and the length of each oligonucleotide probe must be sufficient for sequence-specific hybridization to occur but not so long that fidelity is reduced during synthesis. In some embodiments, the length of the primers range from about 15 to about 40 nucleotides (specifically including 17, 20, 21, 22, 24, 25, 32, 35, and 36), preferably about 15 to about 36 nucleotides, more preferably about 19 to about 25 nucleotides, most preferably about 21 to about 25 nucleotides.

As used herein, a primer refers to a small synthetic single-stranded nucleic acid molecule that anneals or selectively hybridizes to a selected template nucleic acid sequence and serves as a starting point for nucleic acid replication. A forward primer is complementary or substantially complementary to the beginning of a nucleic acid sequence to be replicated and directs sense strand replication. A reverse primer is complementary or substantially complementary to the end of a nucleic acid sequence to be replicated and directs antisense strand replication. Any suitable primers known in the art may be used in accordance with the present invention. Other primers that may be readily constructed or applied in accordance with the present invention by those skilled in the art are contemplated herein.

As used herein, the phrase “selectively (or specifically) hybridizes to” refers to the binding, duplexing, or hybridizing of a nucleic acid molecule to a particular nucleotide sequence only in a sample comprising other nucleic acid molecules under moderate hybridization to stringent hybridization conditions. For selective or specific hybridization, a positive signal is at least about 2 times, preferably about 5 times, more preferably about 10 times the background hybridization. Moderate hybridization conditions are about 10° C. below the thermal melting temperature (Tm) of the probe to about 20° C. to about 25° C. below Tm. Stringent hybridization conditions are about 5° C. below the thermal melting temperature (Tm) of the probe to about 10° C. below Tm.

The hybridization conditions may be less stringent than the conditions exemplified herein. For example, the magnesium chloride concentration, temperature, and the like may be modified according to methods known in the art in order to make the conditions less stringent. It should be noted, however, that the changes in stringency may affect assay sensitivity and specificity. Thus, in some embodiments, the hybridization conditions are stringent hybridization conditions.

As used herein, “substantially complementary” refers to a sequence which is not 100% identically, but specifically hybridizes, to a sequence under moderate, preferably stringent, hybridization conditions.

As used herein, “sequence identity” in the context of two or more nucleic acid molecules, refers to two or more sequences or subsequences that are the same or have a specified percentage of nucleotide bases that are the same (i.e., 70% identity, optionally 75%, 80%, 85%, 90%, 95%, or more identity over a specified region), when compared and aligned for maximum correspondence over a comparison window, or designated region as measured using one of the following sequence comparison algorithms or by manual alignment and visual inspection. The percentage of sequence identity may be calculated by comparing two optimally aligned sequences over the window of comparison, determining the number of positions at which the identical residues occur in both sequences to yield the number of matched positions, dividing the number of matched positions by the total number of positions in the window of comparison (i.e., the window size), and multiplying the result by 100 to yield the percentage of sequence identity.

Methods of alignment of sequences for comparison are well-known in the art. See e.g. Smith & Waterman (1981) Adv. Appl. Math. 2:482; Needleman & Wunsch (1970) J. Mol. Biol. 48:443; and Pearson & Lipman (1988) PNAS USA 85:2444, which are herein incorporated by reference. Alignment may be conducted using computer programs such as GAP, BESTFIT, FASTA, and TFASTA in the Wisconsin Genetics Software Package (Genetics Computer Group, 575 Science Dr., Madison, Wis.), or manually by visual inspection. See also Feng & Doolittle (1987) J. Mol. Evol. 35:351-360; Higgins & Sharp (1989) CABIOS 5:151-153; and Devereaux et al. (1984) Nuc. Acids Res. 12:387-395, which are herein incorporated by reference.

Alternatively, BLAST and BLAST 2.0 algorithms may be used to determine the sequence identity of two or more sequences. See Altschul et al. (1977) Nuc. Acids Res. 25:3389-3402 and Altschul et al. (1990) J. Mol. Biol. 215:403-410, which are herein incorporated by reference. BLAST analyses are publicly available through the National Center for Biotechnology Information at the World Wide Web at ncbi.nlm.nih.gov.

### Cystatin E/M is a Tumor Suppressor

### A. Ectopic Expression of Cystatin E/M Results in Reduced Cell Growth

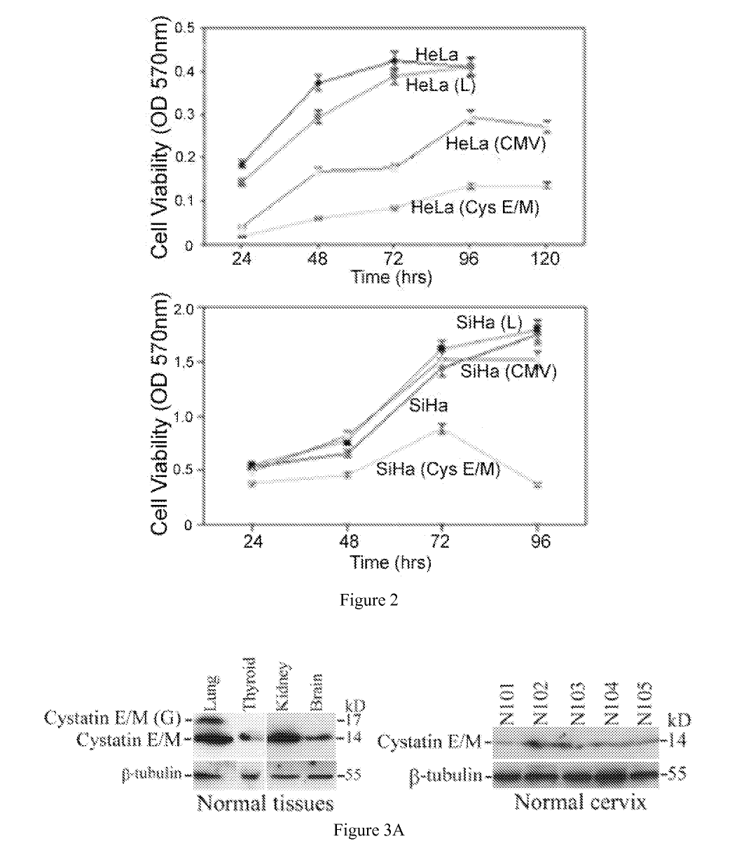

To demonstrate that cystatin E/M expression resulted in cell growth control, the cystatin E/M gene was transfected into HeLa and SiHa cells and assayed for gene expression and cell growth. Expression was assayed by RT-PCR and Western blot analyses. Expression was observed in both the cell lines up to 96 hours post-transfection. Although there was some cell killing seen with the control vector, introduction of the cystatin E/M gene resulted a significant increase in cell growth inhibition in the HeLa cells as shown in FIG. 2. In SiHa cells, the control vector had no effect on cell growth, while cystatin E/M expression slowed down growth. In both cell lines, very few cells survived longer than 96 hours post-transfection, thereby indicating that the expressed cystatin E/M protein strongly suppresses the growth of cancer cells.

### B. Cystatin E/M Expression Reduces Soft Agar Colony Forming Potential of HeLa Cells

Soft agar colony formation assays of HeLa cells transfected with the cystatin E/M gene (HeLa E/M cells) were used to evidence whether the cystatin E/M protein is a tumor suppressor. Western blot studies confirmed the expression of cystatin E/M protein in the HeLa E/M cells. Because there was extensive cell death upon cystatin E/M expression, it was difficult to perform soft agar colony formation assays on isolated clones. Therefore, the assays were carried out on HeLa E/M cells grown in selection medium (Modified Eagle's Medium containing the antibiotic G418 (about 200-800 μg/ml) as the selection agent, Invitrogen, Carlsbad, Calif.) for 10 days. Spherical and irregular colonies were apparent after about 5 days in control cells (HeLa cells transfected with pCMV only). However, visible colonies appeared in the HeLa E/M after about 12 days. Examination of soft agar plates after 21 days showed over a 50%, about 60%, reduction in the number of colonies (P<0.01, Student's t-test) in the HeLa E/M cells. The size of the individual colonies was also reduced in the HeLa E/M cells.

Therefore, the cystatin E/M gene is a tumor suppressor gene and its expression product suppresses the growth of cervical cancer cells.

### C. Cathepsin L Expression

Cervical cancer cell lines and primary tumors were examined to determine whether cystatin E/M has an effect on the expression of cathepsins on cervical cancer cells. There was high level expression of cathepsin L in a variety of cell lines including HeLa, C4-1, HT3, C33A, MS751, SiHa and Caski cells. Cathepsin B was expressed at a reduced level and the expression of cathepsin L2/V was undetectable. Reexpression of cystatin E/M in HeLa E/M cells resulted in lower steady state levels of cathepsin L and cathepsin L expression was undetectable in the media of cells expressing cystatin E/M.

To determine whether cathepsin L is indeed involved in growth promotion in cervical cancer cells and tumors, overexpression studies were performed. Cathepsin L overexpression increased the cell growth and an intermediate growth was observed with the transfection of both the cathepsin L and cystatin E/M genes. These results indicated that cystatin E/M protein downregulates cathepsin L protein levels and results in growth inhibition.

Thus, primary tumors were analyzed for the expression of cathepsin L. Two tumors (#21—invasive squamous cell carcinoma and #22—small cell keratinizing squamous cell carcinoma) that did not express cystatin E/M were found to contain higher levels of cathepsin L in comparison to that of the corresponding normal tissues. These results indicated an inverse relationship between the expression of cystatin E/M and cathepsin L. Also, expression of a lower molecular weight form of cathepsin L was observed in some tumors indicates more extended processing of this form in the absence of cystatin E/M protein.

As disclosed herein, in the presence of cystatin E/M protein, there is very little expression of cathepsin L in CINs. However, cathepsin L is highly expressed in tumor samples.

### Loss of Cystatin E/M Expression

### A. Loss in Cervical Cancer Cell Lines and Primary Tumors

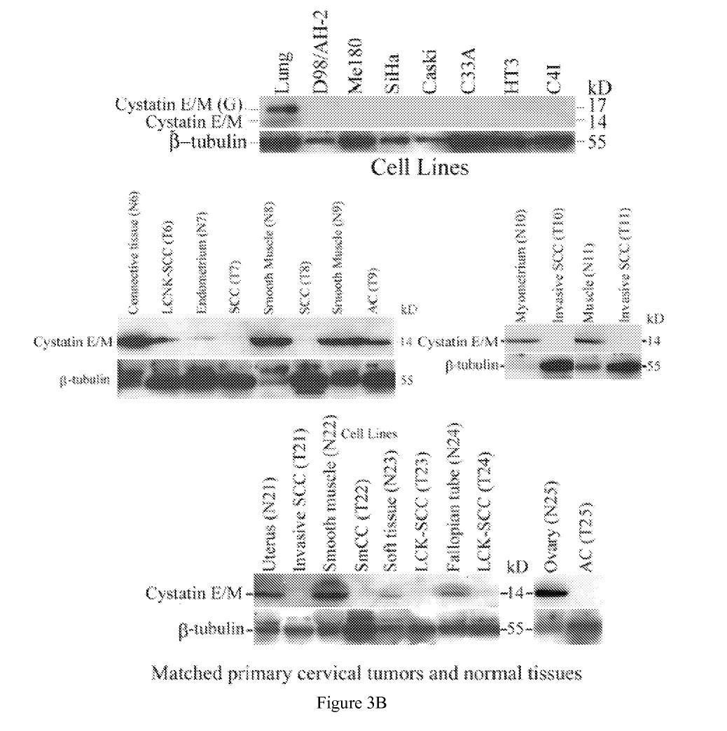

As shown in FIG. 3A, Western blot analysis indicates that cystatin E/M protein is expressed in normal tissues and normal cervix. However, as shown in FIG. 3B, Western blot analysis indicates that cystatin E/M expression was absent in 7 of the cervical cancer cell lines, and 9 of 11 primary tumors. Two tumors (tumors #6 and #9) showed low levels of protein expression (which may represent normal cell contamination). All of the normal (non-cancerous) tissues adjacent to the tumors had high level expression of cystatin E/M.

Therefore, the absence or loss of cystatin E/M protein expression in cervical cells and tissues may be used to indicate that the cells and tissues are cancerous.

Similar to CINs and cervical cancer, it was found that cystatin E/M expression is present in low grade ovarian cancers and cystatin E/M expression is lost in high grade clear cell carcinomas. In addition, it was found that cathepsin L is expressed at lower levels in low grade malignancies and high level expression of cathepsin L is observed in aggressive ovarian cancers. Therefore, in addition to cervical tissues, the compositions and methods of the present invention apply to ovarian tissues.

### B. Somatic Mutation of the Exonic Sequences in Primary Tumors

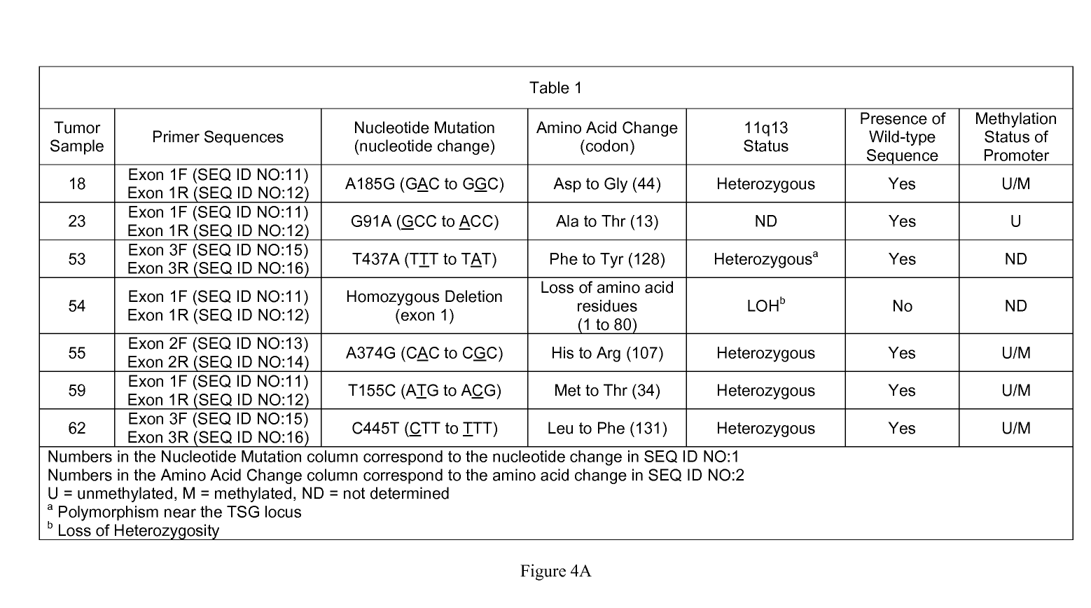

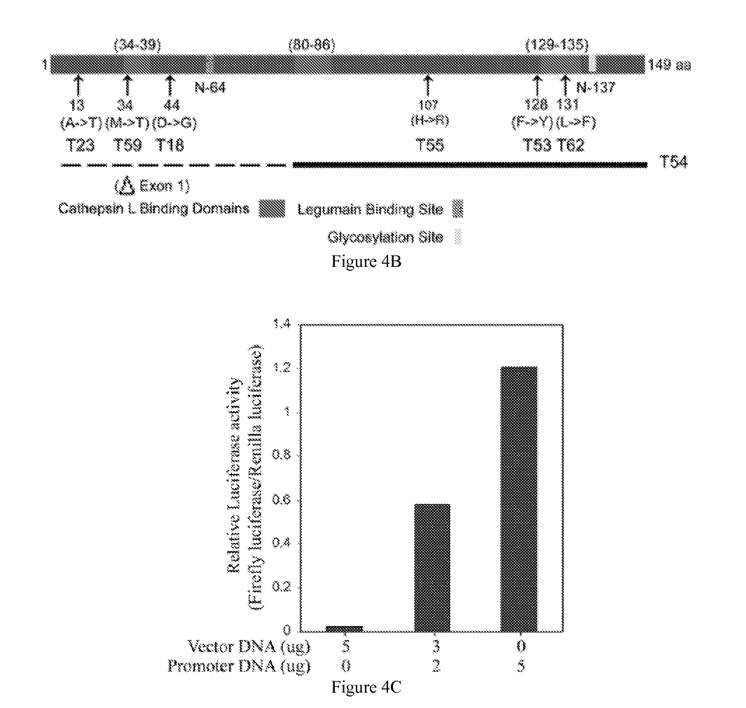

Examination of the three exons of the cystatin E/M gene in four cervical cancer cell lines, 19 primary tumors, and 21 normal controls revealed homozygous deletion of the exon 1 sequence of the cystatin E/M gene in one tumor, various point mutations of the cystatin E/M gene in 6 other tumors, and 2 tumors contained mutations in the cystatin E/M protein at the consensus binding sites for cathepsin L which is a lysosomal protease which is overexpressed in cervical cancer. The results are summarized in Table 1 (FIG. 4A) and FIG. 4B.

Specifically, in order to determine whether the loss of cystatin E/M expression was due to mutations in the exonic sequences of the cystatin E/M gene, the three exons were examined in four cervical cancer cell lines and 19 primary tumors (including two tumors, #11 and #23, that showed a loss of cystatin E/M expression in Western blots). Corresponding normal endometrium for 16 of the tumors, four non involved normal lymphocytes, and a normal lung tissue (a tissue with high level expression of cystatin E/M), totaling 21 normal tissues, were used as controls.

Deletions or point mutations were not observed in any of the 21 normal tissues.

Presence of mutations only in tumor samples in comparison to the matched normal tissues indicated that the mutations are tumor specific. There was homozygous deletion of exon 1 sequences in one of the tumors and a point mutation in six other tumors. The homozygous deletion for exon 1 was observed by the absence of the 369 by PCR product in tumor #54. Although the deletion breakpoints were not determined, additional primer sets showed the deletion to extend at least 260 by upstream of the translation start site. PCR products were seen for the 191 bp, and 268 by sequences of the exons 2 and 3, respectively. EST AW167735 (Srivatsan et al. (2002) Oncogene 21:5631-5642, which is herein incorporated by reference) was used as a control to examine the copy number status of exons 2 and 3. A semiquantitative comparative analysis with the normal tissue revealed the presence of a single copy of exon 3 in tumor #54. Thus, tumor #54 represents a two hit model for loss of cystatin E/M expression where a loss of heterozygosity (LOH) for an exonic sequence is the first event and the homozygous loss of exon 1 sequences as the second event.

The point mutations in six other tumors were missense in nature resulting in the replacement of aspartic acid (D) to glycine (G) in tumor #18, alanine (A) to threonine (T) in tumor #23, phenylalanine (F) to tyrosine (Y) in tumor #53, histidine (H) to arginine (R) in tumor #55, methionine (M) to threonine (T) in tumor #59, and leucine (L) to phenylalanine (F) in tumor #62. See FIG. 4A and FIG. 4B. Three of these mutations were mapped at or near the consensus sites for binding to the lysosomal protease cathepsin L.

For confirming the role of these mutations in the functional inactivation of cystatin E/M, plasmids containing mutated cystatin E/M sequences were used in the transfection studies. Two of the tumor specific mutations (M34T in tumor #59 and L131F in tumor #62) and a third cathepsin L binding site mutation (W135A) were used. See Cheng et al. (2006) J Biol Chem 281:15893-15899, which is herein incorporated by reference.

Hybridization of wild type and mutant cystatin E/M transfected HeLa cells and Western blot analysis showed equal level expression of cystatin E/M in the wild type and the mutants. However, high level expression of cathepsin L was observed in the mutant transfected cells indicating functional inactivation of cystatin E/M in these cells. Immunoprecipitation with the plasmid tag, the FLAG antibody, followed by hybridization to cystatin E/M and Western blot analysis showed the presence of similar levels of cystatin E/M in the wild type and mutant transfected cells. However, there was reduced binding of cystatin E/M protein to cathepsin L in the mutant transfected cells, thereby indicating functional inactivation of cystatin E/M by cathepsin L binding site mutations. The functional inactivation of the cystatin E/M gene with the introduction of these mutations suggests that point mutations and the homozygous deletion represented tumor specific events and not polymorphic changes.

### C. Hypermethylation of the Promoter in Cell Lines and Primary Tumors

Exonic mutations were not detected in the cervical cancer cell lines and in 12 of the primary tumors. Sequencing of the exons has indicated the presence of wild-type allele in the six tumors that contained point mutations. See Table 1, FIG. 4A. Therefore, the promoter region for hypermethylation of CpG islands was examined as an additional reason for inactivation of the cystatin E/M gene.

The +8 to −243 by sequence of the cystatin E/M gene, i.e. TGCTGGGGACTGTCGCTGTCCTCTCCCTCCCCGGGCCAGGTGTGTCCTGGAGGGCAGGGA AGCGTCTTGGCACGCGGGTGCGCGCCGCCCCCTCGGCCTCCTGGGCTCCCTGAACCTCGC AGGACCC [symbol] GGCAACTT [symbol] GAGCCC [symbol] GCCCCAGCTCCAGGC [symbol] G [symbol] GGGG [symbol] GCAT [symbol] G [symbol] GGG [symbol] G T [symbol] GGG [symbol] GGGG [symbol] GGCCCAG [symbol] GGGTAAAAGCTGCGCGGCCGCAAGCTCGGCACTCACGGCTC TGAGGGCTCCG (SEQ ID NO:3) (underlined=primers sequences (forward primer=SEQ ID NO:4 and reverse primer=SEQ ID NO:5), bold=methylation specific PCR sequences, highlight=methylated Cs in HeLa cells, double underline=methylated Cs in Caski cells), was amplified from normal lymphocyte DNA and cloned into a pGL4.16 firefly luciferase vector. HeLa cells were transfected with the vector or the promoter construct, in combination with an internal renilla luciferase control plasmid DNA. The relative luciferase activity with respect to that of renilla luciferase was measured. The results showed a 30- and 60-fold increase in luciferase activity with 2 and 5 μg of the promoter DNA, respectively. See FIG. 4C. Thus, there was a direct relationship between the amount of promoter construct used and the luciferase activity.

Having established the promoter activity, four different cell lines and 18 primary tumors were examined for CpG methylation using the sodium bisulfite protocol. Normal endometrium from 16 of the tumors, four different non-involved normal lymphocytes and a lung tissue were used as controls. The analysis was performed using the sequences spanning the −14 to −151 by region of the promoter (NT_{—}033903.7). Amplification of the 138 by product was observed with the unmethylation specific PCR in 18 of the normal tissues. Similarly, the PCR product was seen for the unmethylation specific primers in 5-AzaCdR treated HeLa, SiHa, and HT3 cell lines and in six primary tumors (including tumors #11, #23, and #36). Untreated HeLa cells yielded the 138 by product with the methylation specific PCR. Cell lines Caski, C41, C33A, three of 21 normal tissues (14%) and 7 of 16 tumors (44%) gave the PCR product with both the unmethylation and methylation specific PCRs. Five other tumors (31%) contained only the methlyation specific product. Thus, there was a significant frequency of methylated promoter in the cervical tumors (12 of 18, 67% in tumors versus 3 of 21, 14% in normals, P<0.01, Student's t-test). Methylation in the three normal endometrial tissues could also represent tumor cell contamination. Four of the tumors contained methylation and point mutation (Table 1, FIG. 4A) indicating possible two hits for the functional inactivation of the cystatin E/M gene.

To confirm promoter hypermethylation in the cell lines, sequencing was carried out on the PCR products cloned into the pTOPO plasmid vector. The characterized sequence showed the absence of C residues in the lymphocyte, 5-AzaCdR treated HeLa, SiHa and HT3 DNAs. All of the C residues were converted into T residues, thereby indicating the presence of an unmethylated promoter in these samples. However, the untreated HeLa cell DNA showed retention of all C residues in the CpG sequences, thereby implying promoter hypermethylation in this cell line. Partial methylation, i.e. retention of some of the C residues of the CpG sequences, was observed for the methylation specific PCR product of the Caski DNA. Thus, the sequencing results confirmed the presence of promoter methylation in two different cell lines, HeLa and Caski. In addition, the data indicated demethylation of the C residues in the HeLa cells after treatment with 5-AzaCdR.

To determine whether 5-AzaCdR treatment resulted in the expression of the cystatin E/M gene, RT PCR analysis was performed. The RT PCR, representing exons 2 and 3, showed expression of the 169 by cystatin E/M product in 24- and 72-hr post-treatment of HeLa cells with either 5-AzaCdR (5 μM, 10 μM, 50 μM) or TSA (150 μM) or in combination (10 μLM 5-AzaCdR, 150 μM TSA). SiHa cells containing unmethylated CpG sequences did not show expression with lower concentrations (5 μM and 10 μM) of 5-AzaCdR, but treatment with 50 μM resulted in a low level expression. High level RNA expression was observed in SiHa cells treated with TSA (150 μM), indicating gene inactivation through a histone deacetylation pathway in this cell line.

To verify that cystatin E/M expression was not limited to RNA synthesis and to identify the cellular location of protein expression, immunofluorescence (IF) was used. Cystatin E/M expression was absent in the untreated HeLa cells. However, cystatin E/M expression was observed in the HeLa cells 24 hours post-treatment with 5 μM 5-AzaCdR. There was uniform expression in the cytoplasm of all cells. The cystatin E/M gene transfected cells on the other hand, showed an increased cytoplasmic expression 48 hours post-transfection. TSA treatment showed a uniform cytoplasmic protein expression in the SiHa cells. Thus, re-expression of the gene by treatment with 5-AzaCdR and TSA in HeLa and SiHa cells, respectively, confirmed promoter methylation and/or histone deacetylation playing a role in the inactivation of cystatin E/M expression in these two cell lines.

Re-expression of cystatin E/M resulted in decreased intracellular and extracellular expression of cathepsin L.

### D. HPV Inactivation

Of the six tumors showing somatic mutations in Table I of FIG. 4A, four (67%) contained HPV (human papilloma virus) sequences, thereby indicating a possible association between HPV and inactivation of cystatin E/M expression. Thus, in order to determine whether HPV coded proteins target cystatin E/M protein for degradation, normal human epidermal keratinocytes (HEK) and those transformed by the E6 gene, the E7 gene, and both the E6 and E7 genes of HPV type 16 were examined for the expression of cystatin E/M.

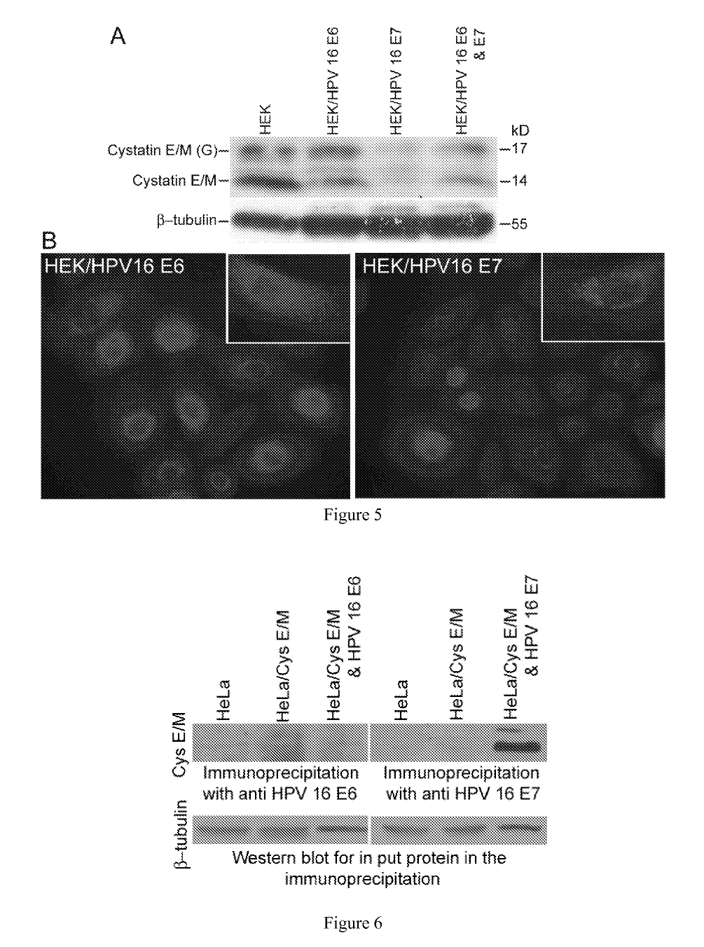

The cells were grown in Epilife (Cascade Biologics, Portland, Oreg.), a medium selected for the growth of keratinocyte cell lines. Western blot analysis using an antibody against cystatin E/M protein (R & D systems, Inc. Minneapolis, Minn.) showed reduced expression of the protein in the cells transformed with the E7 gene and the cells transformed with both the E6 and E7 genes as compared to that of normal keratinocytes (HEK) and cells transformed with the E6 gene. See FIG. 5A. Immunofluorescence with rhodamine staining confirmed reduced cystatin E/M expression in cells transformed with the E7 gene. See FIG. 5B. Higher expression with uniform staining in the perinuclear and cytoplasmic regions was seen in the cells transformed with the E6 gene. Cells transformed with the E7 gene showed reduced punctated perinuclear/cytoplasmic expression possibly representing expression in endosomal compartment of the cells.

To verify whether the reduced expression of cystatin E/M is due to degradation of cystatin E/M by E7 protein, binding studies were performed. HeLa cells were transfected with a plasmid which encodes cystatin E/M alone or in combination with a plasmid which encodes HPV 16 E6 or a plasmid which encodes HPV 16 E7. Forty-eight hours post transfection, proteins prepared from total cell lysates were immunoprecipitated with anti E6 or E7 antibody and Western blotting was carried out on the immunoprecipitates. As shown in FIG. 6, hybridization to the cystatin E/M antibody showed the presence of cystatin E/M protein from the cells transfected with cystatin E/M and HPV 16 E7. These results demonstrate an interaction between E7 and cystatin E/M proteins and that the E7 protein may be responsible for the degradation of the cystatin E/M protein.

### Diagnostics

### A. Loss of Cystatin E/M Expression Evidences Cervical Cancer

To determine whether cystatin E/M expression is suitable for use as a diagnostic, cystatin E/M expression was determined in tissue samples which were clinically characterized as normal cervical epithelium, exhibiting one of the various stages of CIN, and cervical tumors. Specifically, tissue samples of three normal cervical tissues, 14 CINs and 33 invasive tumors were analyzed using immunohistochemical assay methods for cystatin E/M expression.

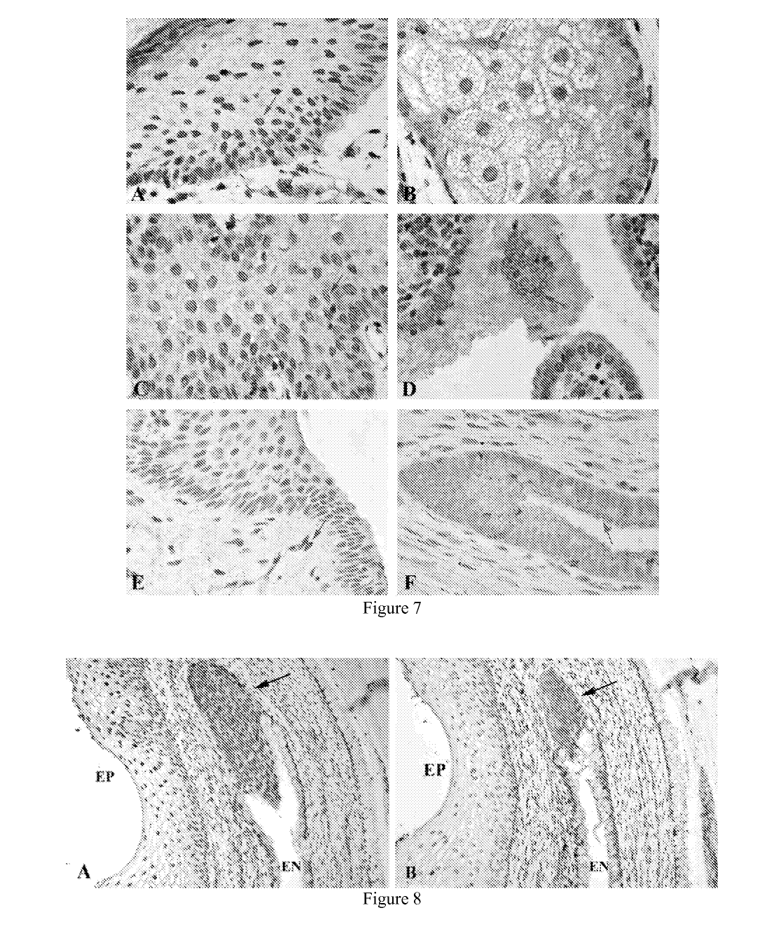

As shown in FIG. 7, cystatin E/M expression was observed in the epithelium of the cervix and endocervical glands of normal cervical tissues. Panels A-D of FIG. 7 show the expression of cystatin E/M in normal skin, sebaceous glands, cervix and endocervix. cystatin E/M expression is localized to the nuclear, perinuclear and to some extent cytoplasm of the cells. Cystatin E/M expression is accompanied by absence of cathepsin L expression in the normal tissues as shown in panels E and F of FIG. 7.

As shown in FIG. 8, cystatin E/M expression was also observed in pre-invasive

CINs and CIN regions of invasive tumors. Specifically, immunohistochemistry was performed on 14 preneoplastic lesions to determine the status of cystatin E/M expression. One sample was clinically classified as CIN 1 and the other 13 were clinically classified as CIN III. Presence of the epithelium in these samples indicated the maintenance of the differentiated state in these lesions. Cystatin E/M expression was seen in the epithelium and endocervix and was accompanied by the absence of cathepsin L in these tissues as shown in FIG. 8.

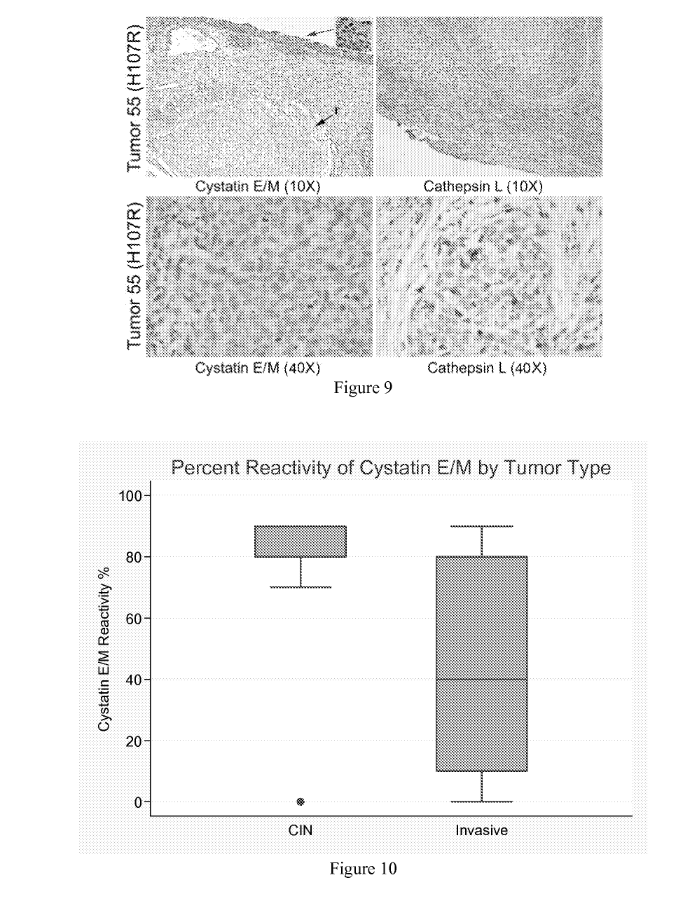

Analysis of 33 primary tumors by immunohistochemistry showed loss of cystatin E/M expression in 26 of the primary tumors. FIG. 9 shows that cystatin E/M expression was lost in aggressive tumors and that the loss of cystatin E/M expression was accompanied by the overexpression of cathepsin L.

These immunohistochemical studies indicate that cystatin E/M expression is lost in the progression from pre-neoplastic to the tumor stage and that the loss of cystatin E/M expression may be used as a diagnostic tool to determine the aggressiveness and extent of tumor invasion in the cervix.

As provided herein, cathepsin L is found to be a target of the cystatin E/M protein in the suppression of tumor development in cervical tissues and that there is an inverse correlation to the expression of cathepsin L and cystatin E/M expression. Thus, in an alternative embodiment, the expression of cathepsin L may be used as a diagnostic tool to determine the aggressiveness and extent of tumor invasion in the cervix. Since cathepsin L is secreted into the extracellular matrix, the amount of cathepsin L outside of a cell may be used to determine the growth potential of the cells.

A statistical evaluation was performed on the expression of cystatin E/M and cathepsin L in CINs and aggressive tumors using the Two-sample Wilcoxon rank-sum (Mann-Whitney) test. Percent reactivity of protein expression was scored from 0-100 and the intensity of expression was scored as 0, 1, 2 and 3 with 3 being the maximum expression.

As shown in FIG. 10, there was a statistically significant (p value=0.0005) difference in the percent reactivity of cystatin E/M expression in CINs as compared to that in invasive tumors.

Fisher's exact test calculation of the intensity of cystatin E/M expression showed a significant difference in the expression in the CINs versus the tumors (Fisher's exact=0.024) as shown in Table 2.

TABLE 2

| | |
|---|---|
| Cystatin E/M | ID Tumor Group |

| Intensity | CIN | Invasive | Total |
|---|---|---|---|
| 0 | 1 | 4 | 5 |
| | 7.14 | 12.12 | 10.64 |
| 1 | 1 | 10 | 11 |
| | 7.14 | 30.30 | 23.40 |
| 1.5 | 0 | 3 | 3 |
| | 0.00 | 9.09 | 6.38 |
| 2 | 4 | 8 | 12 |
| | 28.57 | 24.24 | 25.53 |
| 3 | 8 | 8 | 16 |
| | 57.14 | 24.24 | 34.04 |
| Total | 14 | 33 | 47 |
| | 100.00 | 100.00 | 100.00 |

Fisher's exact = 0.15 (Overall intensity scores)
Fisher's exact = 0.024 (0 + 1 + 1.5 versus 2 + 3)

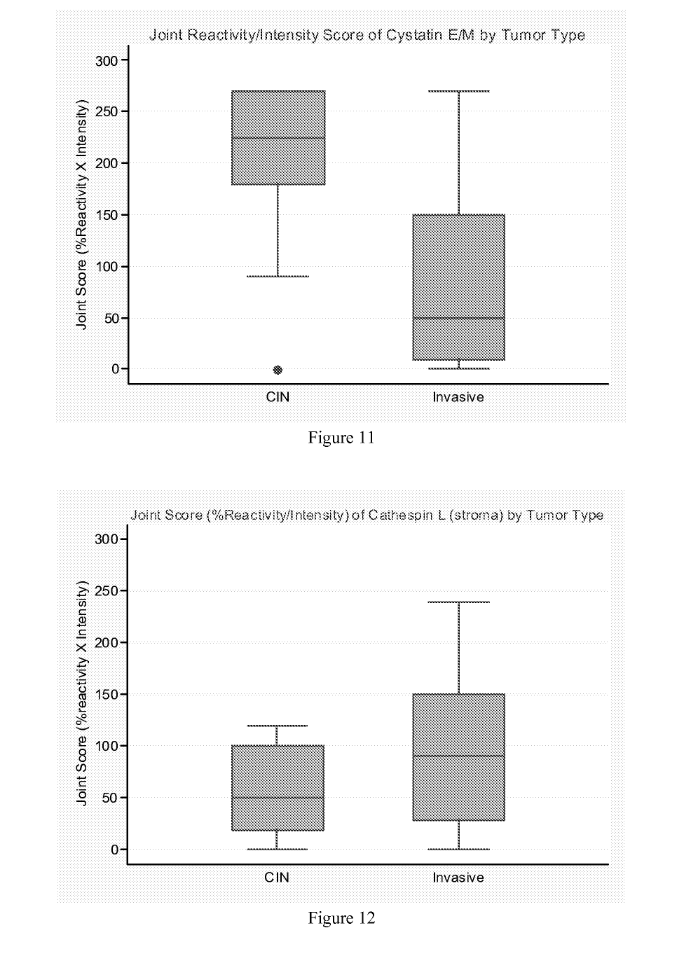

As shown in FIG. 11, a statistical significance (p value=0.0007, Fisher's exact=0.003) was also observed for the joint score of reactivity X intensity of cystatin E/M expression in CINs versus the tumors.

Near statistical significance (p value=0.04, Fisher exact test score=0.16) was observed for the joint reactivity X intensity of cathepsin L expression in the stroma of CINs versus the aggressive tumors as shown in FIG. 12. In this case, lower level expression of cathepsin L in CINs was observed as compared to the higher expression in tumors.

Therefore, in view of these studies, cystatin E/M expression can be used for diagnosing and treating CINs and aggressive cervical tumors. In these embodiments, Western blot hybridization and immunohistochemistry methods may be used to assay the amount of cystatin E/M expression in a sample. Using Western blots, protein isolated from the biopsy specimens is separated on acrylamide gels and detected by hybridization with antibody that specifically recognizes cystatin E/M protein. Using immunohistochemistry, biopsy sections fixed to glass slides are hybridized to cystatin antibody that specifically recognizes cystatin E/M protein and the complex between the cystatin E/M protein and antibody is detected.

As provided herein, high expression of cystatin E/M protein was observed in all the normal tissues and loss of cystatin E/M expression (expression was 5% or less in cervical cancers in comparison the expression in normal cervical epithelial regions) was seen in greater than about 75% of cervical cancers and in 3 of 3 aggressive clear cell ovarian carcinomas (p<0.01%) studied. Therefore, the loss of cystatin E/M expression may be used to diagnose an aggressive phenotype and a treatment regime based on the diagnosis may be prescribed.

As provided herein, the loss of cystatin E/M expression can be caused by a loss of heterozygosity (LOH) for an exonic sequence, homozygous deletion of the exon 1 sequence of the cystatin E/M gene, a mutation causing an amino acid substitution, hypermethylation of the promoter of the cystatin E/M gene, HPV proteins, or a combination thereof. Therefore, primers and probes for sequences disclosed herein and the methods disclosed herein and known in the art may be used to detect whether a subject is at risk for suffering from a cancer associated with a loss of cystatin E/M expression, such as cervical and ovarian cancer.

1. In accordance with the present invention, a deletion of a given exonic sequence in the cystatin E/M gene in a tissue sample may be detected by (a) amplifying the exonic sequence using polymerase chain reaction and primers specific for the sequence, (b) detecting the amount of the amplified exonic sequence (PCR product) of (a) using methods known in the art, e.g. polyacrylamide gel electrophoresis (PAGE) and comparing the amount to a control, e.g. that of a normal tissue from the same individual, and (c) comparing the amount of PCR product of (a) with a genomic sequence localized about 5 to about 100 kb away from the deletion sequence, e.g. EST AW167735. A loss of heterozygosity (LOH) for the given exonic sequence will result in an amount of PCR product that is about half of the amount obtained from normal tissue. Homozygous deletion of the given exonic sequence will result in a little to no PCR product.

2. In accordance with the present invention, a mutation in the cystatin E/M gene causing an amino acid substitution present in a tissue sample may be detected by (a) amplifying the exonic sequences for the cystatin E/M gene using polymerase chain reaction and primers exonic sequences, (b) sequencing the PCR products from (a), and (c) comparing the sequences with a control, e.g. wild-type sequence. In some embodiments, retention of two copies of the genomic sequence, i.e. equal intensity as that of normal tissue, is assayed using PAGE. In some embodiments, the PCR product of (a) are cloned into a plasmid vector, the plasmid clones containing the cystatin E/M exonic sequence are isolated and sequenced.

3. In accordance with the present invention, hypermethylation of the promoter of the cystatin E/M gene in a tissue sample may be detected as follows: (a) using bisulfite to convert cytosine to uracil (cytosine is retained in methylated DNA of tumor tissues) on the nucleic acid obtained from the sample, (b) amplifying the converted nucleic acid sequence from (a) using PCR and methylation and unmethylation specific primers, and (c) detecting thymine residues for unmethylated DNA and cytosine residues for the methylated DNA in the amplified PCR products. In some embodiments, the PCR product of (b) are cloned into a plasmid vector, the plasmid clones are isolated and sequenced.

4. HPV proteins may be assayed using any suitable method known in the art. For example, the proteins from normal and tumor tissues may detected using antibodies which specifically bind HPV E6 and E7 proteins. Such antibodies may be obtained commercially (e.g. Santa Cruz Biotechnology, Santa Cruz, Calif.) or made using methods known in the art.

### B. Cancer Stage and Prognosis

As provided herein, the cystatin E/M gene is expressed in cervical intraneoplasias (CINs), but the expression is reduced or completely lost in later stages of cervical cancer, e.g. in tumors that have lost the morphological characteristics of epithelium. Therefore, the present invention provides methods which detect or measure the amount of the cystatin E/M protein in order to diagnose whether a subject is suffering from a cervical intraneoplasia, e.g. CIN 1-3, or cervical cancer. Cystatin E/M expression may be used to monitor and/or modify a given treatment regime.

### Treatment Regimes

### A. Administration of Cystatin E/M Protein

Since cystatin E/M protein is expressed by all normal tissues, in some embodiments, the present invention provides a method of treating a cancer or inhibiting growth of a tumor which are caused by a loss of cystatin E/M expression or a mutant form of cystatin E/M protein which comprises administering to the subject a therapeutically effective amount of an exogenous cystatin E/M protein. In some embodiments, the exogenous cystatin E/M protein is wild type human cystatin E/M protein. In some embodiments, the exogenous cystatin E/M protein is a recombinant protein. In some embodiments, the exogenous cystatin E/M protein is a functional equivalent of wild type human cystatin E/M protein, i.e. it exhibits a biological activity which is substantially the same as that of the wild type human cystatin E/M protein.

Loss of cystatin E/M expression caused by deletion of an exonic sequence, genetic mutations may be treated by protein replacement therapy, e.g. administering the wild-type cystatin E/M protein to the subject. Alternatively, loss of cystatin E/M expression caused by deletion of an exonic sequence and genetic mutations may be treated by gene therapy, e.g. expressing the wild-type sequence in the subject.

Similar to the treatment of some leukemias, loss of expression due to hypermethylation of the promoter of the cystatin E/M gene may be treated with a demethylating agent such as 5-azacytidine or 5-azadeoxycytidine.

Loss of cystatin E/M expression caused by histone deacetylation may be treated by administration of histone deacetylase (HDAC) inhibitors, such as TSA.

The primers and probes according to the present invention include those which specifically hybridize or amplify the nucleotide mutations, exonic deletions, or amino acid changes set forth in Table 1 (FIG. 4) and as described herein. Other nucleic acid molecules in accordance with the instant invention, include sequences which specifically hybridize to at least 15 contiguous nucleotides of SEQ ID NO:1 containing one of the mutations described herein, e.g. for nucleotide mutation G91A (amino acid change A13T) sequences include GCTGAGCCTGGCCCT (SEQ ID NO:6), CCGCTGGGCCTGACCCT (SEQ ID NO:7), CGCTGGCGCTGAGCCTGGCCC (SEQ ID NO:8), and the like, and complementary sequences thereof for nucleotide mutation T437A (amino acid change F128Y) sequences include CGCTGTGACTATGAGGTCCTTG (SEQ ID NO:9), ACTATGAGGTCTTTG (SEQ ID NO:10), and the like (wherein either T or C may be at nucleotide position 445), and complementary sequences thereof. Thus, in some embodiments, the present invention provides isolated nucleic acid molecules selected from the group consisting of SEQ ID NOs:3-39, preferably SEQ ID NOs:4-39. Such nucleic acid molecules may be used as primers and/or probes. In some embodiments, the present invention is directed to methods of using the nucleic acid molecules of the present invention.

As provided herein, the present invention provides methods for detecting the loss of cystatin E/M expression. In some embodiments, the present invention provides methods for detecting genetic mutations which are associated with a loss of cystatin E/M expression. In some embodiments, cystatin E/M expression levels or the mutations are measured directly (e.g. at the RNA or protein level). In some embodiments, cystatin E/M expression levels or the mutations are detected in tissue samples (e.g. biopsy tissue). In some embodiments, cystatin E/M expression levels or the mutations are detected in bodily fluids (e.g. plasma, serum, whole blood, mucus, and urine). In some embodiments, the presence of a mutation and/or the amount of cystatin E/M expression is used to provide a prognosis to a subject.

In some embodiments, the present invention provides a panel for the analysis of a plurality of cancer markers and the genetic mutations which are associated with a loss of cystatin E/M expression. As used herein, the term “cancer marker” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient. Cancer marker expression may be characterized using any suitable method, including but not limited to, those described herein.

The panel allows for the simultaneous analysis of multiple cancer markers, including the genetic mutations which are associated with a loss of cystatin E/M expression, correlating with carcinogenesis and/or metastasis. For example, a panel may include at least one probe according to the present invention which specifically binds a target molecule which correlates to cancerous tissue, metastatic cancer, localized cancer that is likely to metastasize, pre-cancerous tissue that is likely to become cancerous, and pre-cancerous tissue that is not likely to become cancerous. Depending on the subject, panels may be analyzed alone or in combination in order to provide the best possible diagnosis and prognosis. Any of the probes and primers described herein may be used in combination with each other or with other known or later identified cancer markers.

In some embodiments, the present invention provides an expression profile of cystatin E/M in a tissue or cell, such as cervical or ovarian tissues or cells, at various stages (e.g. normal, benign, pre-cancerous, cancerous) or prognoses (e.g. likely to become cancerous, likely to metastasize). The expression profile can be used for comparison with a sample obtained from a subject. Any suitable method may be utilized, including but not limited to, by computer comparison of digitized data. The comparison data is used to provide diagnoses and/or prognoses to subjects.

As used herein, an “expression profile” refers to the expression levels of a polypeptide in a biological sample for a given cancer and its stages. A standard expression profile for cervical tissue may provide the expression level of a given polypeptide in normal cervical tissue, cervical tissues classified as CIN 1-3 (alternatively LSIL and HSIL based on the Bethesda System), and cancerous cervical tissues in the various stages based on the FIGO or TNM staging system) which may be used to diagnose a subject as having cervical tissue in a particular stage and/or provide a prognosis. In some embodiments, the standard expression profile is generated from pooled samples comprising tissue samples from a plurality of subjects with the same type of tissue, cancer and stage.

The expression profile may be presented as a graphical representation (e.g., on paper or on a computer screen), a physical representation (e.g., a gel or array) or a digital representation stored in a computer readable medium.

In some embodiments, cystatin E/M expression may be detected and measured using any suitable method including Northern blot analysis, enzymatic cleavage, hybridization assays, PCR, RT-PCR and the like.

In some embodiments, cystatin E/M expression may be detected and measured using any suitable method including immunohistochemistry, immunoassays, Western blots, precipitation reactions, agglutination assays, complement fixation assays, immunofluorescence assays, protein A assays, and immunoelectrophoresis assays, and the like.

In some embodiments, a computer-based analysis program is used to translate the raw data generated by the detection assay (e.g. the presence, absence, or amount of a target molecule) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

In some embodiments, the present invention provides kits for the detection and characterization of pre-cancerous tissues and cancers associated with a loss of cystatin E/M expression or mutant cystatin E/M protein such as CINs and cervical cancer. In some embodiments, the kits comprise probes, primers or antibodies specific for a target molecule according to the present invention, in addition to detection reagents and buffers. In some embodiments, the kits contain all of the components necessary to perform a detection assay, including all controls, directions for performing assays, and any necessary software for analysis and presentation of results. In some embodiments, the kits include reagents necessary to assay cathepsin L.

In some embodiments, in vivo imaging techniques are used to visualize the expression of cystatin E/M protein and/or cathepsin L in a subject. Such imaging techniques include radionuclide imaging, positron emission tomography, computerized axial tomography, X-ray or magnetic resonance imaging methods, fluorescence detection, chemiluminescent detection, and the like.

### Materials and Methods

The cervical cancer cell lines, HeLa (D98/AH-2), C41, SiHa, Caski, HT3, C33A, exemplified in the experiments herein were grown in MEM medium with 10% FBS.

Primary cervical tumor tissues and adjacent normal tissues were obtained from the City of Hope National Medical Center as well as from the cooperative human tissue network of the National Institutes of Health. Human tissues were obtained after the approval from the IRB committees of the West Los Angeles VA Medical Center and the City of Hope National Medical Center. A total of 30 tumors were analyzed. Eleven tumors and corresponding normal tissues were used for protein expression by Western blotting, 19 tumors were studied for somatic mutations and 16 tumors were analyzed for promoter hypermethylation. Sixteen of the corresponding normal tissues and four noninvolved lymphocytes were used as controls for the promoter hypermethylation and mutation analyses.

Unless indicated otherwise, all methods were conducted using methods known in the art.

### Polymerase Chain Reaction (PCR)

Three exons of the cystatin E/M gene identified from the genomic sequence (NT_{—}033903.7) were amplified using the following primers (F=forward, R=reverse):

(SEQ ID NO: 11)
exon 1 F: 5′CGGGCGTCGGCGGGGCGGCCC 3′
(SEQ ID NO: 12)
exon 1 R: 5′GGGCCGGGTGTCCCCTCCCAGC 3′
(SEQ ID NO: 13)
exon 2 F: 5′GACCCCTGACCTGCCCCTACC 3′
(SEQ ID NO: 14)
exon 2 R: 5′GGAGGGCTGGGGCTGGAGGAG 3′
(SEQ ID NO: 15)
exon 3 F: 5′GGTCGAGGCTGGGCTCACCCCT 3′
(SEQ ID NO: 16)
exon 3 R: 5′GGGGCAGAAGCGAAGCAGTTGG 3′

The polynucleotides were denatured at 94° C. for 45 sec; annealed using a step down temperature for 45 sec, and the extension performed at 72° C. for 1 min. Step down temperatures from 65° C. for 4 cycles, 64° C. for 7 cycles, 63° C. for 7 cycles, and 62° C. for 20 cycles for exon 1 and from 58° C. to 57° C. to 56° C. for 5 cycles each followed by annealing at 55° C. for 20 cycles for the amplification of exons 2 and 3 were used. A final extension was performed at 72° C. for 2 min. After verification of product synthesis, i.e. size of 369 bp, 191 bp, and 268 by for exons 1, 2, and 3, respectively, on 10% PAGE (PAGE) gels, PCR samples were purified, cloned into the pTOPO vector (Invitrogen Carlsbad, Calif.) and sequenced. At least three different clones were sequenced for each PCR sample.

The following primer pair was also used for amplifying exon 1 (F=forward, R=reverse):

(SEQ ID NO: 17)
CMexon1new F1: 5′GGAGAACTCCGGGACCTGT 3′
(SEQ ID NO: 18)
CMexon1new R1: 5′CAGTCTGTGCTCCCCATCTC 3′

### Reverse Transcription (RT) PCR

RNA was extracted from the cell lines and tissues using Triozol reagent (Invitrogen, Carlsbad, Calif.). RNA was reverse transcribed using the RT kit (Invitrogen, Carlsbad, Calif.). The synthesized cDNA was used in the PCR for the amplification of sequences representing exons 2 and 3 of the cystatin E/M gene. The following primers were used (F=forward, R=reverse):

(SEQ ID NO: 19)
cystatin E/M F: 5′GTACTTCCTGACGATGGAGATG 3′
(SEQ ID NO: 20)
cystatin E/M R: 5′TAGGAGCTGAGAGGAGTTCTG 3′
(SEQ ID NO: 21)
beta actin F: 5′GTCGCCCTGGACTTCGAGCAAGAG 3′
(SEQ ID NO: 22)
beta actin R: 5′CTAGAAGCATTTGCGGTGGACG 3′

For the amplification of the cystatin E/M gene, an initial denaturation at 94° C. for 4 min followed by 32 cycles at 94° C. for 30 sec, 56° C. for 30 sec, and 72° C. for 30 sec were used with a final extension at 72° C. for 2 min. Amplification conditions for beta actin were similar to that of cystatin E/M except that the annealing temperature was 58° C. and the PCR was performed for 30 cycles. PCR products were separated on 8% TBE (50 mM Tris borate pH 8.0, 1 mM EDTA) gels, followed by ethidium bromide staining, and analyzed using the Kodak 1D software.

### Site Directed Mutagenesis

Point mutations representing the cathepsin L binding sites were introduced using the Quikchange II site directed mutagenesis kit following the manufacturer's protocol (Stratagene, San Diego, Calif.). Briefly, cystatin E/M cDNA cloned in the plasmid expression vector, p3XFLAG-CMV-10 (Sigma Chemicals Co., St. Louis, Mo.), was used in a PCR mixture containing oligonucleotides with point mutations as primers and the PCR was carried out using PfuUltra DNA polymerase (Stratagene, San Diego, Calif.). The same sequences in the forward and reverse orientations were used as primers. The primer sequences used in the PCR are as follows (F=forward, R=reverse):

Cystatin E/M M34T F:
(SEQ ID NO: 23)
5′CGG CCG CAG GAG CGC ACG GTC
GGA GAA CTC CGG GAC 3′
Cystatin E/M M34T R:
(SEQ ID NO: 24)
5′GTC CCG GAG TTC TCC GAC CGT
GCG CTC CTG CGG CCG 3′
Cystatin E/M L131F F:
(SEQ ID NO: 25)
5′TGT GAC TTT GAG GTC TTT GTG
GTT CCC TGG CAG AAC 3′
Cystatin E/M L131F R:
(SEQ ID NO: 26)
5′GTT CTG CCA GGG AAC CAC AAA
GAC CTC AAA GTC ACA 3′
Cystatin E/M W135A F:
(SEQ ID NO: 27)
5′G GTC CTT GTG GTT CCC GCG CAG
AAC TCC TCT CAG C 3′
Cystatin E/M W135A R:
(SEQ ID NO: 28)
5′G CTG AGA GGA GTT CTG CGC GGG
AAC CAC AAG GAC C 3′

The underlined letters in the sequences represent the point mutations (as set forth by the mutational designation, e.g. M34T (indicates a threonine instead of a methionine at position 34 of SEQ ID NO:2 prior to the forward or reverse indication). Each PCR product was digested with the restriction enzyme DPnI (Clontech Inc., San Diego, Calif.) to digest the parental DNA and the product containing the mutant plasmid was used for the transformation of super competent DH5a bacterial cells (Invitrogen Inc., Carlsbad, Calif.). The plasmids were purified and sequenced to confirm the presence of the point mutations.

### Promoter Assay

The putative promoter sequence +8 to −243 of the cystatin E/M gene identified by the promoter program available on the internet having the following address hypertext transfer protocol://world wide web.ualberta.ca/_stothard/javascript/cpg_islands.hypertext markup language (wherein hypertext transfer protocol=http, world wide web=www, and hypertext markup language=html) was amplified from a lymphocyte genomic DNA using primers containing Sad and XhoI restriction enzyme sequences at the 5′ end. The following primers were used (F=forward, R=reverse):

REST MET F1:
(SEQ ID NO: 29)
5′CTGAGCTCTGCTGGGGACTGTCGCTGTCCTCT 3′
REST MET R1:
(SEQ ID NO: 30)
5′ATCTCGAGCGGAGCCCTCAGAGCCGTGAGTGC 3′

The PCR product was digested with Sad and XhoI restriction enzymes (Clontech Inc, San Diego, Calif.), purified using a microcon 100 filter (Millipore Corp., Bedford, Mass.) and cloned into the SacI-XhoI site of the firefly luciferase plasmid pGL4.16 (Promega Corporation, Madison, Wis.). Plasmid DNA was sequenced to confirm that the promoter was in the correct read through orientation of the luciferase gene. The promoter construct and the promoterless control pGL4.16 vector were mixed in different ratios to a final DNA amount of 5 μg and transfected into a semiconfluent culture (70-80%) of HeLa cells. The promoter assay was then carried out on cell extracts with the luciferase reagent using the manufacturer's protocol (Promega Corporation, Madison, Wis.).

Other primers used for promoter hypermethylation experiments included the following (F=forward, R=reverse):

MET F3: 5′TAAGAGCCCACGAAGAGCTG 3′ (SEQ ID NO: 31)
MET R3: 5′CCTTGATGATGTGCGTGTCT 3′ (SEQ ID NO: 32)
MMET F1: 5′CATTTATTTCCTGCCGGTGT 3′ (SEQ ID NO: 33)
MMET R1: 5′CCACTTGAGCCAAGGAGTTC 3′ (SEQ ID NO: 34)
MMET F2: 5′CTCCCAAAGTGCTGGGATTA 3′ (SEQ ID NO: 35)
MMET R2: 5′GAGGGCTGTCCAGAATGAAC 3′ (SEQ ID NO: 36)

### Sodium Bisulfite DNA Modification and PCR

DNA samples were treated with sodium bisulfite using the Chemicon CpG genome fast modification kit (Chemicon International, Temecula, Calif.). Primer sequences identified by the primer 3 three program (a freely available web based program to select primers from genomic sequences developed at MIT, Boston Mass.) and spanning −14 to −151 of the cystatin E/M gene promoter were used for the unmethylation and methylation specific PCR (use of specific primers SEQ ID NOs:37-39 to detect CpG methylated bases in tumor DNAs). While a single forward primer, Unmet F: 5′ GGTTTTTTGGGTTTTTTGAATTTTG 3′ (SEQ ID NO:37), was used for both the unmethylation specific PCR and the methylation specific PCR, the reverse primer for the unmethylation specific PCR was Unmet R: 5′ TACCAAACTTACAACCACACAACT 3′ (SEQ ID NO:38) and the reverse primer for the methylation specific PCR was Met R: 5′ TACCGAACTTACGACCGCGCAACT 3′ (SEQ ID NO:39).

For the PCR analysis, 40 ng of bisulfite treated DNA was denatured for 94° C. for 5 min and subjected to a 35 cycle amplification with denaturation at 94° C. for 45 sec, annealing at 59° C. for 45 sec, and extension at 72° C. for 1 min. A final extension was performed at 72° C. for 2 min and the PCR products were cloned and sequenced. At least three different clones were sequenced for each sample.

Treatment With 5′Aza 2-deoxycytidine (5-AzaCdR) or Trichostatin A (TSA)

HeLa and SiHa cells were grown overnight to about 70 to about 80% confluency and treated with 5 μM, 10 μM, 50 μM of 5′Aza 2-deoxycytidine (5-AzaCdR) and/or 150 nM of Trichostatin A (TSA) for up to about 72 hr. Treated cells were used for immunofluorescence or for the extraction of DNA for PCR and total RNA for RT PCR analyses.

### Western Blotting

Proteins were extracted from the cells and tissues using RIPA lysis buffer containing a complete protease inhibitor cocktail (Roche Diagnostics, Indianapolis, Ind.). Proteins secreted into the media were concentrated using a published protocol. See Shridhar et al. (2004) Onocogene 23:2206-2215, which is herein incorporated by reference. Western blotting was performed using about 20 to about 30 μg of denatured proteins on 4-20% SDS acrylamide gels (Invitrogen, Carlsbad, Calif.). Proteins transferred onto nitrocellulose were hybridized to the antibodies (cystatin E/M, cathepsin L from R & D systems, Minn., and beta-tubulin from Santa Cruz Biotechnology, Calif.) using an established protocol. See LoTempio et al. (2005) Clin Cancer Res 11:6994-7002, which is herein incorporated by reference. Immuno-precipitations were carried out using 500 μg of the total protein lysates in RIPA lysis buffer, using the anti-flag antibody (Sigma/Aldrich Chemicals, St. Louis, Mo.). Antibody (10 μg) and protein lysates were incubated overnight at 5° C. followed by incubation with protein G conjugated beads for 3 hours (Santa Cruz biotechnology, Santa Cruz, Calif.). After the incubation, beads were washed four times with lysis buffer and the immune-complexes were eluted using 70 μl of sample buffer. Samples were denatured at 100° C. for 5 min and used for the Western blotting.

### Transfection

Cells (about 1.6×10⁵) were grown in 6-well plates overnight to achieve about 70 to 80% confluency for transfection with pCMV control vector or the cystatin E/M containing plasmid using the Lipofectamine reagent (Invitrogen, Carlsbad, Calif.). Cells were also transfected with cathepsin L cDNA in pCMV vector (Origene Technologies, Rockville, Md.). Twenty-four hours after transfection, cells were used for the cell viability assays at different time periods. Stable transfectants were isolated using zeocin as the selection reagent to a maximum of about 800 μg/ml. Cells grown in selection medium for 10 days (mass culture) were used for soft agar colony assay. Individual clones isolated after extensive selection protocol were used to study the expression levels of cystatin E/M and cathepsin L.

### MTT Assay for Cell Viability

Control and transfected cells (about 4×10⁴) were grown in 24 well tissue culture dishes for 24, 48, 72, and 96 hr. The MTT (3-(4,5-Dimethylthiozol-2-yl)-2,5-diphenyl tetrazolium bromide) assay was carried out following a previously established protocol. See LoTempio et al. (2005).

### Immunofluorescence

Cells were grown overnight on cover slips to semiconfluency (about 70 to 80%) and treated with 5- AzaCdR or TSA, or transfected with plasmid DNAs. Immunofluorescence was performed using the anti cystatin E/M antibody (R & D Systems, Minneapolis, Minn.) following an established protocol. See LoTempio et al. (2005).

### Immunofluorescence for Keratinocyte Cell Lines

Keratinocyte cells expressing HPV16 E6 or E7 were grown in chamber slides to semi-confluencey (about 60-70%). On the day of immunofluorescence, growth medium was removed and cells were washed with cold 1× phosphate buffered saline (PBS). Cells were fixed in 3.5% paraformaldehyde for 30 minutes followed by treatment with freshly prepared 0.2% ammonium chloride, and then with 0.2% Triton X-100 for 5 minutes. Cells were then incubated in 0.5% BSA followed by one hour incubation with primary cystatin E/M antibody (1:100) (R & D systems). After washing with 1×PBS, cells were incubated with anti-mouse antibody tagged with Alexa Fluor 563 (1:400) (Invitrogen, Carlsbad, Calif.) for 30 minutes. Cells were washed with 1×PBS and observed under fluorescent microscope. Staining was recorded at different magnifications.

### Immunohistochemistry

Paraffin embedded transverse sections of normal or tumor tissues (5 μm thick) were collected onto poly-L-lysine-coated slides and allowed to dry. Sections were de-waxed in xylene and rehydrated. Microwave treatment in 10 mM citrate buffer (pH 6) for 3×3 minutes was followed by blockade of non-specific binding by incubation in 0.1 M PBS containing either 3% normal horse or normal goat serum and 0.5% triton X-100 for 30 minutes. Sections were subsequently incubated with cystatin M monoclonal antibody (1:100) (R&D systems). Following extensive rinsing steps in 0.1 M PBS, sections were incubated in ready to use HRP labeled goat anti-mouse for 1 hour at room temperature (Biocare, Concord, Calif.) and subsequent visualization of the reaction product. For negative controls the primary antibody was omitted.

### Soft Agar Colony Assay

Cystatin E/M and control vector transfected mass cultures (grown in zeocin selection medium for 10 days) were trypsinized, and suspended in MEM medium containing 0.1% lukewarm agar at a cell concentration of about 5×10³ cells/ml. The suspension was spread on top of 0.5% solidified agar plates. The agar plates were incubated for 21 days at 37° C. Colonies were stained with 0.001% crystal violet blue, counted, and photographed using a Zeiss microscope.

The examples disclosed herein are intended to illustrate but not to limit the invention.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of diagnosing a subject as having a cancer or identifying a tissue of being cancerous which comprises
assaying the loss of cystatin E/M expression in a biological sample obtained from the subject or the tissue which comprises
(a) detecting the presence of a nucleotide mutation in a cystatin E/M gene obtained from the biological sample which corresponds to nucleotide position 91, 155, 185, 374, 437, or 445 of SEQ ID NO:1, or an exonic deletion;
(b) detecting the presence of an amino acid change in a cystatin E/M protein obtained from the biological sample which corresponds to amino acid position 13, 34, 44, 107, 128, or 131 of SEQ ID NO:2, or a deletion of one or more amino acid residues;
(c) comparing the amount of the cystatin E/M protein or the amount of mRNA which encodes the cystatin E/M protein obtained from the biological sample with a control or a standard;
(d) detecting hypermethylation of the promoter of the cystatin E/M gene obtained from the biological sample;
(e) detecting the presence of a Human Papilloma Viral protein in the biological sample; or
a combination thereof,
wherein a loss cystatin E/M expression of about 80-100%, preferably about 90-100%, more preferably about 95-100% as compared to a control or standard is indicative of the subject having the cancer or the tissue being cancerous.

2. The method of claim 1, wherein the cancer is cervical cancer and the biological sample or tissue is cervical tissue or the cancer is ovarian cancer and the biological sample or tissue is ovarian tissue.

3. The method of claim 1, and further comprising detecting the expression of cathepsin L in the biological sample or tissue sample.

4. The method of claim 3, wherein expression of cathepsin L at a level above normal expression levels is indicative of the tissue being cancerous tissue.

5. An isolated nucleic acid molecule which
(a) specifically binds to a nucleotide region having at least 15 and up to 40 nucleotide bases, and (al) a nucleotide sequence or its complement having SEQ ID NO:1 with at least one nucleotide mutation selected from the group consisting of A185G, G91A, T437A, A374G, T155C, and C445T, or (a2) a genomic sequence which corresponds to the nucleotide sequence or its complement;
(b) encodes at least 5 and up to 12 contiguous amino acid residues of an amino acid sequence having SEQ ID NO:2 with at least one amino acid change selected from the group consisting of D44G, A13T, F128Y, H107R, M34T, and L131F;
(c) specifically binds to a primer sequence that (c1) comprises at least 15 and up to 40 contiguous nucleotide bases of SEQ ID NO:1, a genomic sequence which corresponds to SEQ ID NO:1, or a complement thereof, and (c2) is adjacent to the nucleotide region, or a sequence indicative of encoding SEQ ID NO:1 with at least one amino acid deletion.

6. The isolated nucleic acid molecule of claim 5, wherein the sequence of the nucleic acid molecule is not SEQ ID NO:1 or SEQ ID NO:3.

7. The isolated nucleic acid molecule of claim 5, wherein the sequence of the nucleic acid molecule is SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39.

8. A panel which comprises at least one nucleic acid molecule according to claim 5.

9. A kit comprising at least one nucleic acid molecule according to claim 5.

10. An expression profile which provides the expression levels of cystatin E/M in normal tissue, pre-neoplastic tissue, and cancerous tissue.

11. The expression profile of claim 10, and further comprising the expression levels of cathepsin L in normal tissue, pre-neoplastic tissue, and cancerous tissue.

12. A method of assaying the loss, if any, of cystatin E/M expression in a biological sample which comprises
(a) detecting the presence of a nucleotide mutation in a cystatin E/M gene obtained from the biological sample which corresponds to nucleotide position 91, 155, 185, 374, 437, or 445 of SEQ ID NO:1;
(b) detecting the presence of an amino acid change in a cystatin E/M protein obtained from the biological sample which corresponds to amino acid position 13, 34, 44, 107, 128, or 131 of SEQ ID NO:2;
(c) comparing the amount of the cystatin E/M protein the amount of mRNA which encodes the cystatin E/M protein obtained from the biological sample with a control or a standard;
(d) detecting hypermethylation of the promoter of the cystatin E/M gene obtained from the biological sample;
(e) detecting the presence of a Human Papilloma Viral protein in the biological sample; or
a combination thereof.

13. A method of treating a subject suspected of having a cancer which comprises
(a) diagnosing the subject as having the cancer according to claim 1; and
(b) administering a treatment to the subject accordingly.

14. The method of claim 13, and further comprising
(c) monitoring the effect of the treatment by assaying the amount of cancerous tissue in the subject and/or measuring the amount of cystatin E/M protein in a sample obtained from the subject.
